(19) United States
(12) Patent Application Publication
(10) Pub. No.: US 2005/0226915 A1
(43) Pub. Date: Oct. 13, 2005

(54) Method for manufacturing and packaging oral patches with rounded edges

(75) Inventor: Jeffrey T. Haley, Mercer Island, WA (US)

Correspondence Address: GRAYBEAL, JACKSON, HALEY LLP, 155 - 108TH AVENUE NE, SUITE 350, BELLEVUE, WA, 98004-5901, US

(73) Assignee: Oramelts Corporation, a Washington corporation

(21) Appl. No.: 11/157,054

(22) Filed: Jun. 20, 2005

Related U.S. Application Data
(63) Continuation-in-part of application No. 10/287,843, filed on Nov. 5, 2002.
Application No. 10/287,843 is a continuation-in-part of application No. 10/236,289, filed on Sep. 4, 2002, now abandoned.
(60) Provisional application No. 60/344,577, filed on Dec. 28, 2001.

(51) Int. Cl. A61K 9/70

(52) U.S. Cl. 424/443

(57) ABSTRACT

A method of manufacturing and packaging discs made of hydrophilic gums, such as adherent, soluble oral patches for delivering topical medication in the mouth. The hydrophilic gums are mixed with water and deposited as blobs onto base sheet material such as thermoplastic. The blobs on the sheets are then dried with air convection, causing the blobs to shrink in height, forming tapered discs with rounded edges. Before being deposited, the mixture may be heated to above an activation temperature that is below boiling, and then, after being deposited, the mixture is allowed to cool to form a gel. The base sheets serve as packaging and a lid sheet may be adhered to the base sheet, such as by heat sealing with a hot platten pressing both sheets against an anvil. The platen or the anvil (or both) has recesses that prevent the discs from being pressed during sealing. The sealed package is then cut to size, such as by die cutting.

As a continuation in part, this application claims priority from PCT/US03/34549 filed Oct. 29, 2003 and from U.S. Ser. No. 10/287,843 filed Nov. 5, 2002 which, for the United States, is a CIP of 10/236,289 filed Sep. 4, 2002 and claims priority from 60/344,577 filed Dec. 28, 2001.

### BACKGROUND

For treatment of health problems in the mouth or throat, people have for centuries held in their mouths a composition containing herbal or other medication for topical application. The oldest name for such a composition, derived from Latin and previously from Greek, is “troche”. A modem form of troche is the cough drop, so named because it was formed by “dropping” hot, viscous, sugar-based candy onto a sheet or into a mold where it cools to form the troche. Another modem form of troche is the throat “lozenge”, so named because it was in the shape of a diamond (like on playing cards), which is the meaning of the word “lozenge”.

To keep troches from moving about in the mouth, or to achieve higher concentrations of medication at a particular spot in the mouth than troches can deliver, or to occlude a lesion in the mouth, adherent oral patches have been developed. As used herein, the word “patch” does not include preparations that move about the mouth rather than adhering in one place, such as cough drops, throat lozenges, or troches. Nor does it include preparations that do not hold together as a single item when held in the mouth such as preparations of powder, liquid, paste, viscous liquid gel, or a tablet or troche that crumbles into a powder or paste when chewed or placed in saliva. The most significant differences between an oral patch as used herein and other forms of oral medicinal topical preparations such as troches are that an oral patch is designed to (1) release medication into the mouth over a relatively long period of time, such as 30 minutes or more, (2) be at least mildly adherent so that it can be placed in a preferred location and not be dislodged by gravity or gentle movement, and (3) remain in the mouth as a single item that will not spread to be in a plurality of locations in the mouth at one time.

U.S. Pat. No. 6,139,861 issued to Mark Friedman describes a method for making oral patches called “mucoadhesive erodible tablets” by pressing hydrophilic polymer gums such as carboxymethylcellulose, hydroxymethylcellulose, polyacrylic acid, and carbopol-934 into tablets. Alternatively, it is known to make mucoadhesive patches by forming materials into a thin sheet and die cutting patches out of the sheet. Both the tablet pressing process and the die cutting process produce a patch having uncomfortably thick and square edges. A preferred shape would be tapered, thickest in the center, and tapering to a thin but not sharp or square, rounded edge.

### SUMMARY OR THE INVENTION

In one aspect, the invention is a method for making an adherent oral patch that is tapered, thickest in the middle and tapering to a thin but not sharp or square, rounded edge. The oral patch is made by mixing hydrophilic polymers such as food gums or other hydrophilic polymers with water to form a viscous mixture, depositing blobs of the mixture onto a sheet or mold, and then allowing the blobs to dry. The polymers form a structural network. As they dry, the blobs get much thinner but retain most of their diameter. Because the blobs were thickest in their centers upon being deposited, they remain thickest in their centers after drying. Because the blobs had rounded edges upon being deposited, they still have rounded edges after drying, even though the edges may be quite thin. This process produces thin discs made of hydrophilic polymers with a shape that feels quite comfortable in the mouth when adhered to a mucosal surface in the mouth.

In another aspect, the invention is a method for manufacturing and packaging discs of hydrophilic polymers. As described above, blobs are deposited onto a flat sheet or a sheet that is formed into a mold with recesses for the blobs. The sheet or mold may be thin plastic or aluminum serves as packaging for delivery of the discs to a consumer. To complete the package, a top sheet of lidding material is sealed to the bottom sheet or mold. The entire package may then be die cut to a preferred size and shape.

In additional aspects, the invention is an oral patch made by the method described above, or a package of discs comprising hydrophilic polymers made by the process described above, or an oral patch tapered from thickest in the center to the edge, with a rounded edge.

### BRIEF DESCRIPTION OF THE DRAWINGS

FIG. 1 shows a diagram of steps in the invented methods.

FIG. 2 shows a side view of a deposited blob of mixture.

FIG. 3 shows a side view of a blob that has been dried to form a thin disc with the preferred taper and rounded edges.

### DETAILED DESCRIPTION

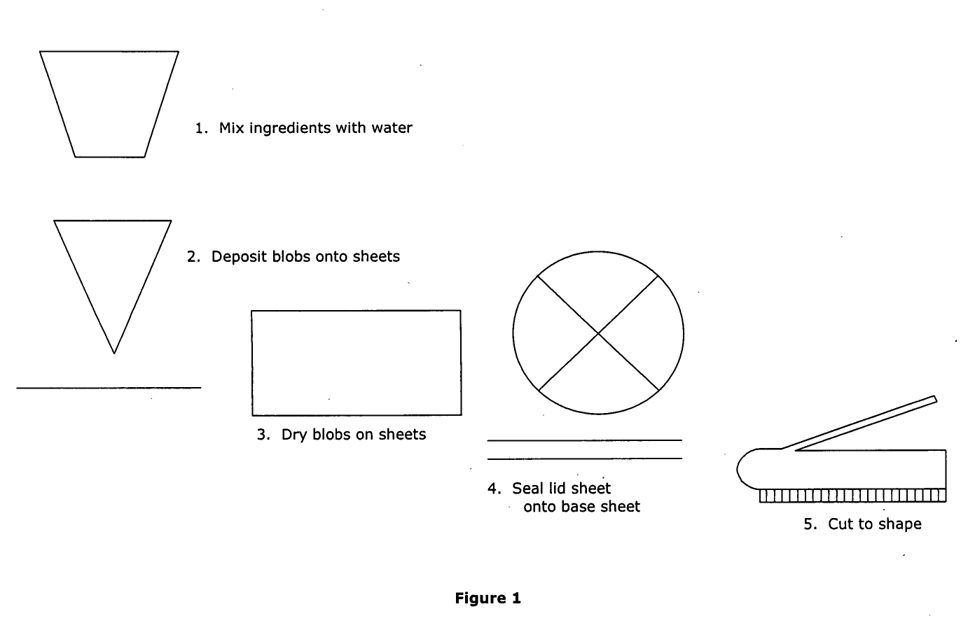

As shown in step 1 in FIG. 1, the process begins by mixing ingredients including water and hydrophilic polymers, such as food gums or synthetic polymers such as carboxymethylcellulose, hydroxymethylcellulose, polyacrylic acid, or carbopol-934. Other ingredients that should be released as the finished product dissolves or erodes, such as flavoring, coloring, a medication, or and ingredient to aid erosion/dissolution, may be added. To retain flexibility in the finished product, a non-evaporating plasticizer, such as glycerol (glycerin) may be added within the structural network formed by the polymers. However, the more glycerol is added the less adherent the oral patch will be.

For ease of manufacturing, it is convenient if the polymers form a thermo gel having a melting temperature higher than human mouth temperatures. This allows the entire mixture to be a liquid at temperatures far above human mouth temperatures and allows the deposited blob to become strong by cooling the mixture such that the thermo gel forms a strong blob by a gelation process. The temperature at which the gel forms can be lower than human mouth temperatures, provided the temperature at which it melts again is higher than human mouth temperatures.

Readily available materials that form such a gel include many of the food gums, such as agar, in various forms, carrageenan, in most of its forms, particularly kappa carrageenan, konjac gum, locust bean gum, and xanthan gum. All of these materials form a thermo gel that is sufficiently elastic or plastic or a combination thereof to feel soft in the human mouth if it is adequately hydrated.

However, gelation after deposition is not required. The blobs can remain tacky and viscous while they are drying.

Synthetic hydrogels may be used. Protein-based hydrogels are usually prepared using proteins extracted from natural sources, but they may be synthesized, such as with diblock copolypeptide amphiphiles, as taught by Nowak, et. al, “Rapidly Recovering Hydrogel Scaffolds From Self-Assembling Diblock Copolypeptide Amphiphiles”. Nowak, A. P.; Breedveld, V.; Pakstis, L.; Ozbas, B.; Pine, D. J.; Pochan, D.; Deming, T. J. Nature, 2002, 417, 424-428. The use of synthetic materials allows adjustment of copolymer chain length and composition. Synthetic hydrogels may also be made from polysaccharides and synthetic block copolymers which form thermoreversible gels and allow the solubilisation of hydrophobic medications for controlled release, as taught by Williams, P A, at the Centre for Water Soluble Polymers, North East Wales Institute, Plas Coch, Mold Road, Wrexham, Wales.

Instead of forming the mixture with a true hydrogel, the mixture may be formed with a hydrophilic complex carbohydrate, such as cellulose, pectin, starch, maltodextrin or other polysaccharides, which for purposes of this explication, are considered to be hydrophilic polymers. Forming of hydrated network structures out of such materials is well known in the candy making industry for making gummy candies. Or the mixture may be formed with a combination of a true hydrogel and a complex carbohydrate.

For desirable adhesion and erosion characteristics in an adherent oral patch, the mixture may include a hydrophilic polymer that is liquid at human mouth temperatures. Collagen molecules, such as gelatin rendered from animal protein, such as from pork or cattle skin or from fish, serve very well as this ingredient. Collagen molecules tend to adhere very well to the tissues of the mouth lining which, themselves, are collagen molecules. The collagen molecules may be partially hydrolyzed, making them shorter and lower in molecular weight, in the form of commercially available gelatin.

Commercially available gelatins are graded according to “bloom strength” which refers to the strength of the gel that is formed. Gelatin with a higher bloom strength (made with longer collagen molecules) is preferred for the adherent oral patch because it also has a higher viscosity in liquid form. The high viscosity in liquid form prevents the gelatin molecules from escaping the mixture substantially faster than the mixture erodes, and the high viscosity better retains molecules of medication for slow release. The highest commercially available bloom strength, 250, is preferred.

As or after the mixture is prepared, it may be heated to reduce viscosity or to activate or prevent gelation or to minimize growth of bacteria during manufacturing. For the latter purpose, a temperature at or above 140 degrees F. is preferred. To avoid complications of boiling, the mixture should be kept below the boiling temperature, which because molecules are dissolved in the water, may be about 215 degrees F. Within this range, the temperature may be selected by manufacturing considerations. A temperature range of 160 degrees to 180 degrees has been found effective.

As shown by step 2 in FIG. 1, the mixture is then deposited onto sheets or molds. Depositors used in the candy making industry are suitable, such as depositors made by NID of Australia. One process is to form a well-hydrated mixture at temperatures above the activation temperature and below the boiling temperature of water so that water does not boil off and yet the hydrogels are fully activated for gelling when the product is cooled. In this process, the mixture can be formed of a combination of a true hydrogel such as xanthan gum with locust bean gum or with konjac gum and a complex carbohydrate such as cellulose or pectin or starch. For a patch with the medication licorice root extract, an effective ratio by units of weight is 9690 units water, 1640 units gelatin, 724 units licorice extract, 960 units cellulose, and 274 units of xanthan gum mixed with konjac gum heated to between 140 and 200 degrees F.

The mixture is poured or squirted into open top molds which may be a flat sheet. Open top molds may be formed by pressing a plug into powdered starch such as cornstarch or may be formed in a tray for packaging the products such as thermo formed PVC or PET or a cold press laminate of aluminum and PVC with a thin layer of polyamide for strength. The molds may be plastic lined, in which case the plastic becomes a part of the final packaging.

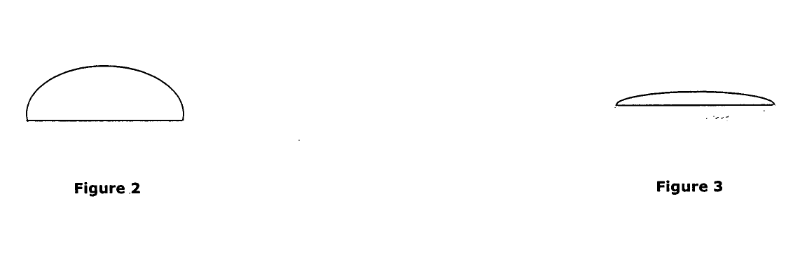

A suitable size for an oral patch is 0.8 grams of mixture poured onto the mold or sheet. When deposited onto a flat sheet, each blob has a cross section as shown in FIG. 2. The height of each blob relative to its width is a function of the viscosity of the mixture as it is deposited. The width and height of each blob are adjusted by changing viscosity and the amount of material in each blob to achieve a preferred shape as shown in FIG. 2.

If the blobs are deposited in powdered starch, the starch absorbs some of the excess water and the blobs are further dried in a drying room before being removed from the starch and packaged.

For cost reasons, it is advantageous to deposit the blobs directly onto a thermoplastic sheet that can be used a part of the final package. Suitable materials include, nylon, polystyrene, polyethylene, and polypropylene. A suitable thickness is 0.030 inches. The sheets can then be placed in a drying room and then covered with a sheet of lid stock such as metal foil or thin plastic coated with sealant. The process can be done on individual sheets on individual trays of any suitable size for running through the depositor or can be done with continuous roll sheet material which runs through a long drying chamber.

If the oral patches are deposited onto a tray (or sheet on a tray), the tray is stored in a drying room until the oral patches lose a suitable amount of moisture, as shown by step 3 in FIG. 1. A suitable method of drying is to expose the blobs to room temperature and humidity for 3 days with or without convection. If convection is used, the sheets can be closely stacked. Refrigeration type dehumidifiers can lower the humidity to 30% which is quite sufficient. The drying process can be taken to any desired level of dryness, such as dry enough that microorganisms will not grow.

The drying process converts a blob with a profile shown in FIG. 2 to a thin disc with a profile as shown in FIG. 3. For an oral patch, the shape shown in FIG. 3 is preferred. The thickest point is in the center and it gently tapers toward the edges until the final edge is rounded, not square or sharp. The ratio of maximum thickness to diameter can be adjusted by changing the viscosity of the mixture. A preferred embodiment is 1-3 millimeters thick in the center and 8-14 millimeters in diameter with tapering as shown in FIG. 3.

To prevent the discs from gaining or losing moisture, they may be packaged with a hermetic seal. Alternatively, the oral patches may be packaged with a base or lidstock film that allows moisture to pass so moisture can easily be added to or removed from the oral patches without removing them from the packaging. If the packaging film is a barrier to germs, this allows the oral patches to remain sterile and not grow mold even when they are moist. Effective films are cellophane, polystyrene, poybutadiene, polyamide, Tyvek (matted polyethylene threads) and expanded films such as Goretex. Polyamide with a thickness of 0.7 mil to 1.0 mil is effective. Allowing such a package to sit for a day or two with a few drops of water on the package is sufficient to hydrate the oral patch inside. Conversely, allowing the package to sit on a shelf in a dry room for one to three days is sufficient to dry out the oral patch.

As shown at step 4 in FIG. 1, the base sheets are sealed with a film or foil lid that is adhered by conventional sealing techniques. Hot or cold sealant may be used by printing the sealant in a suitable pattern onto the base sheet or onto the lid stock. Or the lid stock can be coated with heat sealant and a platen with recesses can press the sheets together onto an anvil where the anvil or the platen is heated. The recesses prevent the dried discs from being squashed or excessively heated. The platen and anvil may be in the form of rollers with either or both of them heated, or one of them may be flat while the other is flat except for recesses. The platen can be pressed with a hydraulic press or a pneumatic press.

If the lid stock is aluminum foil, that discs may be released from the packaging by pushing them through the foil. Pressing flat lid stock onto a flat sheet with discs between them requires that the base sheet or the top sheet (or both) flexes (stretches) to avoid wrinkles. The aluminum will be most stretchy if it is not tempered. Although standard aluminum lid stock coated with heat sealant is high temper, it is preferable to order custom foil with zero temper.

As shown by step 5 in FIG. 1, the sheets that have been sealed together are cut into cards of suitable shape, such as by die cutting. Cards that easily fit in a pocket are preferred, such as 2.3 inches by 3.4 inches.

The entire package may be sterilized with gamma radiation or heat and pressure in a retort.

While particular embodiments of the invention have been described above, the scope of the invention should not be limited by the above descriptions but rather limited only by the following claims.

1. A method for manufacturing an adherent oral patch for delivering topical medication in a human mouth over time, comprising:
(a) mixing a mixture comprising the medication, ingredients for forming a hydrophilic porous network with a low to moderate rate of disintegration in saliva that remains a solid at human mouth temperatures, and water;
(b) depositing the mixture onto a mold; and
(c) drying the mixture, thereby causing the ingredients for forming a network to form a hydrophilic porous network as a unitary solid structure having the medication within its pores.

2. The method of claim 1 wherein, before depositing the mixture, it is heated to a gelation activation temperature and the network is formed by a process of gelation with cooling.

3. The method of claim 1 further comprising drying the patch and then enclosing it in a package.

4. The method of claim 1 wherein the network is comprised of a thermogel having a melting temperature higher than human mouth temperatures.

5. The method of claim 1 wherein the mixture also comprises molecules of a hydrophilic polymer that is liquid at human mouth temperatures and the hydrophilic polymer forms a thermoreversible gel that is a gel at room temperatures.

6. The method of claim 5 wherein the hydrophilic polymer is comprised of protein.

7. The method of claim 6 wherein the hydrophilic polymer is gelatin rendered from animal tissue.

8. The method of claim 1 wherein the mold is formed of powdered starch.

9. The method of claim 1 wherein the mold is a flat, rigid sheet.

10. The method of claim 1 wherein the mold comprises a sheet of plastic.

11. The method of claim 10 further comprising sealing a cover onto the sheet around the patch.

12. A method for forming and packaging discs comprising hydrophilic polymers, the method comprising:
(a) mixing the hydrophilic polymers with water to form a mixture;
(b) depositing blobs of the mixture onto a base sheet of packaging material;
(c) drying the blobs by exposing the base sheet to convection of air to form discs; and
(d) sealing a lid sheet of packaging material to the base sheet at places of sealing contact surrounding each disc.

13. The method of claim 12 wherein the blobs are deposited using a depositor machine that deposits a plurality of blobs onto the base sheet in a row, all at one time.

14. The method of claim 12 wherein the base sheet is flat, the lid sheet is flat, and at least one of the sheets is flexed to seal to the other at the places of sealing contact around each disc.

15. The method of claim 12 further comprising, after the base sheet and the lid sheet are sealed together, cutting both sheets together to form a final package.

16. The method of claim 12 wherein the cutting process is by die cutting.

17. The method of claim 12 wherein the sealing is by heating with a hot platen pressing the sheets together at the places of sealing contact against an anvil to melt a sealing layer of sealant on at least one of the sheets.

18. The method of claim 12 wherein the lid sheet is comprised of aluminum.

19. The method of claim 18 wherein the aluminum is of low or zero temper.

20. The method of claim 12 wherein the mixture is heated before it is deposited to a temperature between 140 degrees F. and 215 degrees F.

21. An adherent oral patch made by the method of claim 1.

22. A sealed package of discs comprising hydrophilic polymers made by the method of claim 12.

23. An adherent oral patch that is tapered from center to edge, thickest in the center, and tapering to a thin, rounded edge.
